(19) United States
(12) Patent Application Publication
(10) Pub. No.: US 2021/0154109 A1
(43) Pub. Date: May 27, 2021

(54) MAGNESIUM DELIVERY SYSTEM

(71) Applicant: NFuse, LLC, Owings Mills, MD (US)

(72) Inventors: Emily Offutt ZOLLINGER, Owings Mills, MD (US); Ann Offutt AHL, Kennett Square, PA (US)

(73) Assignee: NFuse, LLC, Owings Mills, MD (US)

(21) Appl. No.: 17/164,527

(22) Filed: Feb. 1, 2021

Related U.S. Application Data
(62) Division of application No. 16/144,364, filed on Sep. 27, 2018.
Application No. 16/144,364 is a continuation-in-part of application No. 15/615,033, filed on Jun. 6, 2017, now Pat. No. 10,117,894.
(60) Provisional application No. 62/348,516, filed on Jun. 10, 2016.

(51) Int. Cl. A61K 8/19; A61Q 15/00; A61K 8/41; A61K 8/34

(52) U.S. Cl. CPC A61K 8/19; A61K 8/345; A61K 8/41; A61Q 15/00

(57) ABSTRACT

A roll-on product, including magnesium hydroxide; propanediol; glycerin and Glycine soja (soybean), is advantageously used as a deodorant and/or for transdermal magnesium delivery to the human body. Combinations of a magnesium source (especially magnesium hydroxide), with propanediol, and in some formulations with glycerin and Glycine soja (soybean) are advantageous for transdermal delivery of magnesium to the body via a roll-on product, a stick, a spray, a lotion, gel, cream, etc. Magnesium deficiency is addressed via products easily used by an ordinary consumer for dermal application.

### FIELD OF THE INVENTION

The invention relates to ameliorating magnesium deficiency in a human, and more particularly, transdermal delivery of magnesium to a human body especially via underarm skin.

### BACKGROUND OF THE INVENTION

Over 50% of Americans' diets are deficient in magnesium. For many people, dietary intake of magnesium is insufficient due to low intake of magnesium rich foods, low magnesium content in drinking water, and unpleasant laxative effects of orally ingested magnesium.

Bathing in Epsom salts has been known conventionally as an approach in which the body can receive some magnesium through bathing, but such a bathing-based approach is time-consuming, and can be impractical or impossible for, or rejected by, some people.

Magnesium chloride sprays have been known to cause itching and burning on the skin which impedes their popularity and effectiveness for widespread use remedying magnesium deficiency.

There has been an unmet need for easy-to-use, non-oral products readily and quickly useable by people deficient in magnesium.

### SUMMARY OF THE INVENTION

The present inventors recognized that there was a problem of there being, conventionally, no magnesium-delivery product in a form of a personal care product useable at least once daily, and they solved that problem by inventing magnesium permeation technology, beginning with magnesium permeation technology that optimizes absorption of magnesium to the body through the underarms.

An objective of this invention is to provide a transdermal delivery system which administers an alkaline magnesium compound to the underarm skin such as through a roll-on, a stick, a spray, a gel, a cream, a lotion, etc. The process of the alkaline magnesium compound reacting with moisture, acids, and salts present on and around the underarm skin causes magnesium ions to be released and available for absorption into the skin.

Another objective of the invention is to provide technology in which a magnesium compound (such as magnesium hydroxide, magnesium oxide, magnesium sulfate, magnesium chloride, or a combination thereof) is used alone or in combination with one or more ingredients that increase permeation of magnesium ions into the underarm skin.

A further objective of the invention is to provide methodology for administering, to the body, a therapeutic dose of elemental magnesium at a ratio between 5-60%, more preferably 10-60%, or total composition weight.

In a preferred embodiment, the invention provides a deodorant that controls odor and delivers magnesium to the body.

The invention in another preferred embodiment provides a transdermal magnesium supplement for use under the arms.

In another preferred embodiment the invention provides a transdermal magnesium supplement, such as for use in health care and wellness fields, for physicians and health care professionals to prescribe to populations struggling with magnesium deficiency.

The invention also includes a preferred embodiment that provides a transdermal magnesium supplement in health care and wellness fields for physicians and health care professionals to prescribe to enhance effectiveness of conventional treatments for various health conditions including but not limited to asthma, depression, diabetes, arrhythmia and heart failure, migraine headache, osteoporosis, gout and restless leg syndrome.

In another preferred embodiment the invention provides a combined magnesium replacement system and deodorant for chemotherapy patients who are at risk for hypomagnesemia due to side effects of chemotherapy drugs.

The invention in another preferred embodiment provides a combined magnesium replacement system and deodorant for athletes during and after workouts to restore magnesium levels and speed muscle recovery.

In another preferred embodiment the invention provides a transdermal magnesium supplement for premenopausal and menopausal relief.

The invention in another preferred embodiment provides a natural deodorant for people concerned about possible link of aluminum, parabens, phthalates, synthetic fragrances, and other harmful chemicals to breast cancer and Alzheimer's disease.

In another preferred embodiment, the invention provides a back pain reliever and/or provides muscle pain relief and/or provides neck pain relief.

In another preferred embodiment, the inventions provides a sleep aid that can be applied one hour prior to bedtime.

The invention in a preferred embodiment provides a roll-on product, comprising a container inside which is disposed a quantity of a solution comprising: water; a magnesium component (such as, e.g., magnesium hydroxide; magnesium sulfate; magnesium chloride; combinations thereof; etc.); propanediol; and glycerin and Glycine soja (soybean), such as, e.g., inventive roll-on products wherein the solution consists of water, magnesium hydroxide, propanediol, glycerin and Glycine soja (soybean), and the one or more plant extracts, and only the solution is inside the container; inventive roll-on products wherein the solution has an intradermal magnesium delivery characteristic of a quantity of magnesium being delivered to a human to whom the solution has been applied to dermis; inventive roll-on products wherein the solution has the intradermal magnesium delivery characteristic when the solution has been applied underarm; and other inventive roll-on products.

In another preferred embodiment, the invention provides a solution for transdermal magnesium delivery, comprising: water, a magnesium compound (such as, e.g., magnesium hydroxide; magnesium sulfate; magnesium chloride; combinations thereof; etc.) and propanediol; such as, e.g., an inventive solution applicable to human dermis via roll-on from a roll-on container; an inventive solution applicable to human dermis via spraying from a container; an inventive solution applicable to human dermis via a solution-soaked patch; an inventive solution applicable to human dermis via a lotion; inventive solutions and compositions applicable to human dermis via solution-delivery selected from the group consisting of: roll-on from a roll-on container; spraying from a container; a solution-soaked patch; a lotion; a cream and a gel; inventive solutions further comprising glycerin and Glycine soja (soybean); and other inventive solutions and compositions.

The invention in another preferred embodiment provides a method of transdermal delivery of magnesium hydroxide, comprising swiping a human underarm with a roll-on and thereby delivering onto the dermis a solution comprising water, magnesium hydroxide, propanediol, and glycerin and Glycine soja (soybean) (such as, e.g., a swiping step wherein swiping a right underarm and a left underarm of a human delivers about 300 mg magnesium hydroxide onto the dermis of the human).

In another preferred embodiment, the invention provides a roll-on product, comprising a container inside which is disposed a quantity of a solution comprising: water; propanediol; and glycerin and Glycine soja (soybean), such as, e.g., inventive roll-on products further comprising a magnesium compound (such as, e.g., magnesium hydroxide; magnesium chloride; magnesium sulfate; combinations thereof; etc.).

The invention in another embodiment provides a roll-on product, comprising a solid stick comprising: a magnesium compound (such as, e.g., magnesium hydroxide; magnesium sulfate; magnesium chloride; combinations thereof; etc.); propanediol; and glycerin and Glycine soja (soybean).

In another preferred embodiment, the invention provides a lotion, comprising a magnesium compound (such as, e.g., a magnesium compound selected from the group consisting of magnesium hydroxide; magnesium chloride and magnesium sulfate) and propanediol, such as, e.g., lotions further comprising glycerin and Glycine soja (soybean), and other inventive lotions.

The invention in another preferred embodiment provides a gel product for application onto human skin, comprising magnesium and propanediol, formulated in a form of a gel.

### DESCRIPTION OF A PREFERRED EMBODIMENT OF THE INVENTION

For the inventive transdermal magnesium delivery, it will be appreciated that a critical ingredient in any formulation is always a magnesium component. The magnesium component, and indeed each and every component used in the formulation to be used for transdermal magnesium delivery to the body, must be safe for application to human skin. Examples of a magnesium component are, e.g., magnesium hydroxide; magnesium sulfate; magnesium chloride; combinations thereof; etc. Magnesium hydroxide is preferred for use as the magnesium component, such as, e.g., a magnesium hydroxide-containing formulation that is deliverable via a roll-on product; a magnesium hydroxide slurry (such as, e.g., a magnesium hydroxide slurry in which magnesium content to water content is in a range of about 30% Mg:70% H₂O to 70% Mg: 30% H₂O in the slurry, preferably at least 50% Mg (and 50% or less H₂O) in the slurry, more preferably in a range of about 55% Mg:45% H₂O to about 65% Mg:35% H₂O in the slurry, most preferably, at least 60% Mg: 40% (or less) H₂O in the slurry such as, e.g., approximately 60% Mg: 40% H₂O by weight); comprising magnesium hydroxide in a form of, or derived from FloMag® magnesium hydroxide slurry; magnesium hydroxide particles having a median particle size of about 3.0 micron; magnesium hydroxide particles having a size in a range of 1.0-4.0 micron; a magnesium hydroxide slurry characterized by specific surface area of about 10 m²/g; a magnesium hydroxide slurry characterized by specific surface area in a range of 9-20 m²/g; etc.

Flomag® (Flomag H® (commercially available from Martin Marietta) is a magnesium hydroxide slurry that is most preferred to use for an inventive transdermal magnesium delivery formulation. Flomag H® has the following characteristics: On a slurry basis, the Mg(OH), contained is 7.7 lb/gal typically, with 8.0 maximum and 7.0 minimum. On a dry solids basis: the Mg(OH)₂ is 98.8 wt % typically, 98.5% minimum, with CaO typically 0.6 wt %, maximum 0.8%; SiO, typically 0.20 wt %, maximum 0.35; Fe, typically 0.10 wt %, 0.21 maximum. Flomag H® has a median particle size of 3.0 micron typically, maximum 4.0, minimum 1.0, a specific surface area of 10 m²/g typically, 20 maximum and 9 minimum, and is characterized by 3.21 acres/gallon typically, 3.5 maximum and 3.0 minimum.

An example of a roll-on product is, e.g., a roll-on product comprising a container and a rolling ball that occupies an opening of the container.

In a case of delivery to the skin via roll-on container, a solution of Flomag® preferably is further combined with one or both of a propanediol component, and a glycerin and Glycine soja (soybean) component.

A magnesium component (such as, e.g., magnesium hydroxide; magnesium sulfate; magnesium chloride; combinations thereof; etc.) also can be formulated into a spray delivery system, a stick delivery system, a lotion delivery system, a gel delivery system, etc. Combining the magnesium component (such as, e.g., magnesium hydroxide; magnesium sulfate; magnesium chloride; combinations thereof; etc.) with one or both of a propanediol component, and a glycerin and Glycine soja (soybean) component, is preferred for all transdermal delivery systems, such as solutions used in roll-on containers, solutions used in spray delivery containers, solids used as sticks, lotions, gels, etc.

A preferred example of a glycerin and Glycine soja (soybean) component for use in the invention is a composition comprising glycerin in a range of about 64-82% and Glycine soja in a range of about 18-36% (such as LysoFix® liquid—glycerin and Glycine soja—product #018047 manufactured by Kemin Industries Inc.). The most preferred example of the glycerin and Glycine soja is LysoFix®.

A preferred example of a propanediol component for use in the invention 1-3 propanediol, such as Zemea® propanediol manufactured by Dupont Tate & Lyle BioProducts.

The present inventors sometimes use the term “Triangle Ingredients” herein to mean a combination of (1) a magnesium component; (2) a propanediol component and (3) a glycerin and Glycine soja (soybean). Transdermal application of the Triangle Ingredients, variously formulated, is most preferred for delivering magnesium to the body, including use of formulations comprising only the Triangle Ingredients and formulations comprising the Triangle Ingredients with one or more other optional ingredients.

An amount of magnesium hydroxide or magnesium component in an inventive formulation preferably is in a range of 10-70% by weight, more preferably in a range of 45-55% by weight.

An amount of propanediol in an inventive formulation preferably is in a range of 10-30% by weight, more preferably in a range of 17.5-22.5% by weight.

An amount of glycerin (and) Glycine soja (soybean) seed extract in an inventive formulation preferably is in a range of 1-10% by weight, more preferably 3.5-7.5% by weight.

Examples of a ratio of magnesium hydroxide:glycerin (and) Glycine soja (soybean) seed extract:propanediol in formulations according to the invention are, e.g., in a range of about 9:4:1 to 11:4:1, in a range of about 10:3.5:1 to 10:4.5:1, etc. A most preferred example of a ratio of magnesium hydroxide:glycerin (and) Glycine soja (soybean) seed extract:propanediol in formulation is 10:4:1.

Examples of ingredients optionally contained in formulations for transdermal delivery of magnesium according to the invention are, e.g., essential oils (such as, e.g., lemon, eucalyptus, lavender, Mentha piperita (peppermint) oil, Rosmarinus offinalis (rosemary) leaf oil, Cymbopogon flexuosus (lemongrass) oil, combinations thereof, etc.); seed extract; plant extract; Matricaria recuita (chamomile); flower extract; heptyl undecylenate; acacia senegal gum; sodium bicarbonate; xanthan gum; combinations thereof; etc.

Preferably, a solution to be used for transdermal delivery of magnesium according to the invention avoids, or minimizes, usage of aluminum, parabens, phthalates, artificial preservatives, propylene glycol, and synthetic fragrance. Most preferably, a solution to be used for transdermal delivery of magnesium according to the invention contains no aluminum, no parabens, no phthalates, no artificial preservatives, no propylene glycol, no synthetic fragrance.

Examples of a delivery system for transdermal delivery of magnesium according to the invention are via, e.g., a roll-on, a lotion, a gel solid, a cream (such as, e.g., a shave cream, a blemish cream, etc.), a body wash, a face cleanser, a spray (such as, e.g., a spray deodorant, a body spray, etc.), a powder (such as, e.g., a foot powder, a powder deodorant, etc.), a paste (such as, e.g., a toothpaste, etc.), a mouthwash, etc., with a roll-on, a lotion, and a gel solid being preferred delivery systems.

The invention may be appreciated with reference to the following examples, without the invention being limited to these examples.

### Example 1 (Transdermal Delivery Systems that Administer an Alkaline Magnesium Compound with a pH in the Range of 9 to 11 to the Underarm Skin)

### Example 1A

A method for a transdermal delivery system which administers the alkaline magnesium compound to the underarm skin through one of the following: a roll-on, a stick, a spray, a gel, or a cream. The process of the alkaline magnesium compound reacting with moisture, acids, and salts present on and around the underarm skin causes magnesium ions to be released and available for absorption into the skin.

### Example 1B

A transdermal delivery system wherein the magnesium compound is magnesium hydroxide, magnesium oxide, magnesium sulfate, magnesium chloride, or a combination thereof. The preferred magnesium hydroxide is a hydrolyzed slurry with a ratio of magnesium to water greater than 25%.

### Example 1B1

A preferred formulation includes Flomag®, a hydrolyzed magnesium hydroxide slurry (60% magnesium hydroxide to 40% water) manufactured and sold by Martin Marietta using a patented manufacturing and blending process that results in a chemical reaction to create a highly stable solution with superior surface spread and reactivity.

### Example 1B2

Another preferred formulation includes Flogel®, a low viscosity, magnesium hydroxide gel (29-33% magnesium hydroxide to 67-71% water) manufactured and sold by SPI Pharma.

### Example 1C

Methods according to Examples 1-1B2, wherein the elemental magnesium is at a ratio between 10-60% of total composition weight.

### Example 1D

Transdermal delivery of magnesium according to Examples 1-1A, wherein the absorption of magnesium through the underarm is enhanced using a transdermal delivery system comprising one or more of: (a) active transdermal delivery agent, first means of transport of magnesium through skin; (b) solvent and permeation enhancer, second means for enhancing the permeation of magnesium through the underarm skin; (c) essential oils, third means for enhancing permeability of skin; (d) fourth means for binding the magnesium compound with (a) the active transdermal delivery agent, (b) a solvent and permeation enhancer and (c) essential oils with permeation enhancing properties into a single transdermal delivery system applies to the underarm skin.

### Example 1E

A transdermal delivery system, including as a means for active transdermal delivery one or more of the following vegetable oils: Lysofix®, a soybean seed extract, a sunflower oil, jojoba oil, coconut oil, almond oil, ground nut oil, shea butter oil, corn oil, babassu oil, castor oil, Clark A oil, cotton seed oil, linseed oil, mustard oil, olive oil, palm oil, peanut oil, safflower oil, sesame oil, soybean oil, sunflower-seed oil, and wheat germ oil.

### Example 1E1

A preferred formulation includes Lysofix®, a soybean seed extract manufactured and sold by Kemin Industries. Lysofix® has been found by the inventors to be superior to other permeation enhancers which are currently commercially available.

### Example 1F

A transdermal delivery system, including for enhancing the permeation of magnesium through the underarm skin one or more of the following: propanediol, propylene glycol (1,2-propanediol) and butylene glycol (1,3-1,4-butanediol).

### Example 1G

A transdermal delivery system, including for enhancing the permeation of magnesium through the underarm skin one or more of the following essential oils: peppermint, eucalyptus, clove, ylang ylang, wintergreen, tulsi, rose, black cumin, tea tree, lemongrass, grapefruit, orange, lemon, bergamot, tangerine, basil, nutmeg, thyme, geranium, cinnamon, bay leaf, rosemary, camphor, niaouli, and carraway.

### Example 1H

A transdermal delivery system for magnesium delivery, in which is include a water soluble gum of one or more of the following: agar, arabic, arabic senegal, carob, CMC, carrageenans, ghatti, guar, karaya, kadaya, locust bean, tragacanth, xanthan gum.

### Example 2 (Early Formulations

### Example 2A

### Water 10-85%

### Mg(OH)₂ 10-60%

### Aloe Vera 0-40%

### Glycerin 5-15%

### Sodium Bicarbonate 0-3%

### Xanthan Gum 0-1%

### Essential Oils 0-2%

### Example 2B

### Water 10-85%

### MgO 10-60%

### Aloe Vera 0-40%

### Glycerin 5-15%

### Sodium Bicarbonate 0-3%

### Xanthan Gum 0-1%

### Essential Oils 0-2%

### Example 2C

### Water 10-85%

### Mg(OH)₂ 5-50%

### MgO 5-50%

### Aloe Vera 0-40%

### Glycerin 5-15%

### Sodium Bicarbonate 0-3%

### Xanthan Gum 0-1%

### Essential Oils 0-2%

### Example 2D

### Water 10-85%

### Mg(OH)₂ 1-50%

### MgSO₄ 0-10%

### MgO 1-50%

### Aloe Vera 0-40%

### Glycerin 5-15%

### Sodium Bicarbonate 0-3%

### Xanthan Gum 0-1%

### Essential Oils 0-2%

### Example 2E

### Flomag® 15-90%

### MgSO₄ 0-10%

### Water 0-50%

### Aloe Vera 0-40%

### Glycerin 5-15%

### Sodium Bicarbonate 0-3%

### Xanthan Gum 0-1%

### Essential Oils 0-2%

### Example 2F

### Flogel® 30-90%

### MgSO₄ 0-10%

### Water 0-50%

### Aloe Vera 0-40%

### Glycerin 5-15%

### Sodium Bicarbonate 0-3%

### Xanthan Gum 0-1%

### Essential Oils 0-2%

### Example 2G

### Water 25-75%

### MgSO₄ 20-50%

### Mg(OH)₂ 0-20%

### Alcohol 0-40%

### Witch Hazel 0-45%

### Aloe Vera 0-35%

### Essential Oils 0-2%

### Example 3 (Preferred Formulations

### Example 3A

### Water 10-90%

### Mg(OH)₂ 10-90%

### LysoFix® 1-10%

### Example 3B

### Flomag® 90-99%

### LysoFix® 1-10%

### Example 3C

### Flogel® 90-99%

### LysoFix® 1-10%

### Example 3D

### Water 10-90%

### MgO 10-90%

### LysoFix® 1-10%

### Example 3E

### Water 10-90%

### MgO 1-50%

### Mg(OH), 1-50%

### LysoFix® 1-10%

### Example 3F

### Water 10-90%

### Mg(OH), 10-90%

### Propanediol 10-70%

### LysoFix® 1-10%

### Example 3G

### Water 10-90%

### Mg(OH), 10-90%

### Propanediol 10-70%

### LysoFix® 1-10%

### Essential Oils 0-2%

### Example 3H

### Water 0-50%

### Flomag® 15-90%

### Propanediol 10-70%

### LysoFix® 1-10%

### Essential Oils 0-2%

### Example 31

### Water 0-50%

### Flogel® 30-90%

### Propanediol 10-70%

### LysoFix® 1-10%

### Essential Oils 0-2%

### Example 3J

### Water 10-90%

### Mg(OH), 10-90%

### Propanediol 10-70%

### LysoFix® 1.5-10%

### Arabic Senegal Gum 0-1%

### Xanthan Gum 0-1%

### Essential Oils 0-2%

### Example 3K

### Water 10-90%

### Mg(OH), 10-90%

### Propylene Glycol 10-70%

### LysoFix® 1.5-10%

### Arabic Senegal Gum 0-1%

### Xanthan Gum 0-1%

### Essential Oils 0-2%

### Example 3L

water 10-90%
magnesium hydroxide 10-90%
propanediol 10-70%
glycerin & Glycine soja (soybean) seed extract 2-10%
essential oils 0-2%

### Example 3M

water 10-90%
magnesium hydroxide 10-90%
propanediol 10-70%
glyercin & Glycine soja (soybean) seed extract 2-10%
sodium bicarbonate 0-5%
essential oils 0-2%

### Example 4 (Most Preferred Formulations

### Example 4A

### Water 33.5-43.5%

### Mg(OH)₂ 25-35%

### Propanediol 17.5-22.5%

### LysoFix® 3.5-5%

### Emollient 2.5-5%

### Sodium Bicarbonate 0.5-1%

### Gum 0.5-1.5%

### Essential Oils 0-2%

### Example 4B

### Water 15-20%

### FloMag® 45-55%

### Propanediol 17.5-22.5%

### LysoFix® 3.5-5%

### Emollient 2.5-5%

### Sodium Bicarbonate 0.5-1%

### Gums 0.5-1.5%

### Essential Oils 0-2%

### Example 4C

### Water 36-46%

### Flogel® 63-73%

### Propanediol 17.5-22.5%

### LysoFix® 3.5-5%

### Emollient 2.5-5%

### Sodium Bicarbonate 0.5-1%

### Gums 0.5-1.5%

### Essential Oils 0-2%

### Example 4D

water 10-90%
magnesium hydroxide 10-90%
propanediol 10-70%
essential oils and plant extracts 0-20%

### Example 4E

water 10-90%
magnesium hydroxide 10-90%
propanediol 10-70%
glycerin & Glycine soja (soybean) seed extract 2-10%
essential oils and plant extracts 0-20%

### Example 4F

water 10-90%
magnesium hydroxide 10-90%
propanediol 10-70%

### LysoFix® 0.5-7.5%

essential oils and plant extracts 0-20%

### Example 4G

water 10-90%
magnesium hydroxide 10-90%
propanediol 10-70%

### LysoFix® 0.5-7.5%

emollient 0-10%
essential oils and plant extracts 0-20%

### Example 4H

water 5-20%

### FloMag® 45-55%

### Propanediol 17.5-22.5%

### LysoFix® 3.5-7.5%

emollient 0-10%
essential oils 0-15%

### Example 41

water 5-20%

### FloMag® 17%-60%

### Magnesium Chloride 5-40%

### Propanediol 17.5-22.5%

### LysoFix® 3.5-7.5%

emollient 0-10%
essential oils and plant extracts 0-20%

### Example 4J

water 5-20%

### FloMag® 17%-60%

### Magnesium Sulfate 5-40%

### Propanediol 17.5-22.5%

### LysoFix® 3.5-7.5%

emollient 0-10%
essential oils and plant extracts 0-20%

### Example 4K

water 5-20%

### FloMag® 17%-30%

### Magnesium Chloride 20-35%

### Propanediol 17.5-22.5%

### LysoFix® 3.5-7.5%

emollient 0-10%
essential oils and plant extracts 0-15%

### Example 4L

water 5-20%

### FloMag® 17%-30%

### Magnesium Sulfate 20-35%

### Propanediol 17.5-22.5%

### LysoFix® 3.5-7.5%

emollient 0-10%
essential oils and plant extracts 0-15%

### Example 4M

### FloMag® 50%

### Water 0-18%

### Propanediol 20%

### LysoFix® 5%

### LexFeel Natural 5%

essential oils and plant extracts 1-20%

### Example 4N

### FloMag® 50%

### Water 10-18%

### Propanediol 20%

### LysoFix® 5%

### LexFeel Natural 5%

essential oils 2-10%

### Example 5 (Rosemary-Mint)

A rosemary-mint magnesium roll-on having no aluminum, no zinc salts, no parabens, no phthalates, no propylene glycol, no synthetic fragrance, no preservatives, having as ingredients:

water
magnesium hydroxide
propanediol
glycerin (and) Glycine soja (soybean) seed extract
Matricaria reculita (chamomile) flower extract
heptyl undecylenate
acacia senegal gum
sodium bicarbonate
Mentha piperita (peppermint) oil
Rosmarinus offinalis (rosemary) leaf oil
xanthan gum
Cymbopogon flexuosus (lemongrass) oil

### Example 5A (unscented)

An unscented magnesium roll-on having no aluminum, no zinc salts, no parabens, no phthalates, no propylene glycol, no synthetic fragrance, no preservatives, having as ingredients:

magnesium hydroxide
water
propanediol
glycerin (and) Glycine soja (soybean)
heptyl undecylenate

### Sodium Bicarbonate

xanthan gum

### Acacia Gum

### Example 5B (Citrus)

A citrus magnesium roll-on having no aluminum, no zinc salts, no parabens, no phthalates, no propylene glycol, no synthetic fragrance, no preservatives, having as ingredients:

magnesium hydroxide
water
propanediol
glycerin and Glycine soja (soybean)
heptyl undecylenate

### Chamomile Extract

### Acacia Gum

### Grapefruit

### Sodium Bicarbonate

### Sweet Orange

xanthan gum

### Lemongrass

### Geranium

### Example 5C (Lavender)

A lavender magnesium roll-on having no aluminum, no zinc salts, no parabens, no phthalates, no propylene glycol, no synthetic fragrance, no preservatives, having as ingredients:

magnesium hydroxide
water
propanediol
glycerin and Glycine soja (soybean)
heptyl undecylenate
lavender
acacia gum
sodium bicarbonate
clary sage
xanthan gum
bergamot
lemongrass

### Example 6 (Relief of Back Pain)

A formulation comprising the Triangle Ingredients was used transdermally for relieving back pain. Transdermal application preferably is onto the back where the pain is experienced.

### Example 6A (Relief of Muscle Aches)

A formulation comprising the Triangle Ingredients was used transdermally for relieving muscle aches. Transdermal application is preferably onto the skin near where the muscle aches are experienced.

### Example 6B (Relief of Neck Pain)

In this example, a formulation comprising the Triangle Ingredients is used for relieving neck pain. Transdermal application is preferably onto the skin of the neck.

### Example 7

In this example, a formulation for use in a roll-on product comprises one or more of: seed extract;

Matricaria recuita (chamomile);
flower extract;
heptyl undecylenate;
acacia senegal gum;
sodium bicarbonate;
Metha piperita (peppermint) oil;
Rosmarinus offinalis (rosemary) leaf oil;
xanthan gum; and
Cymbopogon flexuosus (lemongrass) oil.

### Example 8

A formulation for use in a roll-o product comprising (by weight):

water in a range of about 15-20%
magnesium hydroxide in a range of about 45-55%
propanediol in a range of about 17.5-22.5%
glycerin and Glycine soja (soybean) in a range of about 3.5-5%
plant-derived components in a range of about 3.5-9.5%

### Example 8A

The formulation of Example 8, comprising (by weight):

### Flomag® 49.5-50%

propanediol 17.5-22.5%
glycerin and Glycine soja (soybean) 3.5-5%

### Example 9 (Unscented Formulation)

A formulation for a roll-on product consisting of (by weight):

| | |
|---|---|
| FloMag ® | 49.5% |
| Water | 18.5% |
| Zemea ® | 20.0% |
| Lysofix ® | 5.0% |
| Lexfeel Natural | 5.0% |
| Sodium Bicarbonate | 1.0% |
| Keltrol | 0.3% |
| Acacia Gum | 0.7% |

### Example 10 (Rosemary Mint Formulation)

A formulation for a roll-on product consisting of (by weight):

| | |
|---|---|
| FloMag ® | 50.0% |
| Water | 12.7% |
| Zemea ® | 20.0% |
| Lysofix ® | 5.0% |
| Lexfeel Natural | 5.0% |
| Chamomile Extract | 5.0% |
| Acacia Gum | 0.7% |
| Peppermint | 0.4% |
| Rosemary | 0.3% |
| Sodium Bicarbonate | 0.5% |
| Keltrol | 0.3% |
| Lemongrass | 0.1% |

### Example 11 (Citrus Formulation)

A formulation for a roll-on product consisting of (by weight):

| | |
|---|---|
| FloMag ® | 50.00% |
| Water | 11.50% |
| Zemea ® | 20.00% |
| LysoFix ® | 5.00% |
| LexfeelNatural | 5.00% |
| Chamomile Extract | 5.00% |
| Acacia Gum | 0.70% |
| Grapefruit | 0.70% |
| Sodium Bicarbonate | 0.50% |
| Sweet Orange | 0.50% |
| Keltrol | 0.30% |
| Lemongrass | 0.50% |
| Geranium | 0.40% |

### Example 12 (Lavender Formulation)

A formulation for a roll-on product consisting of (by weight):

| | |
|---|---|
| FloMag ® | 50.0% |
| Water | 16.9% |
| Zemea ® | 20.0% |
| LysoFix ® | 5.0% |
| LexfeelNatural | 5.0% |
| Lavender | 0.9% |
| Acacia Gum | 0.7% |
| Sodium Bicarbonate | 0.5% |
| Clary Sage | 0.3% |
| Keltrol | 0.3% |
| Bergamot | 0.2% |
| Lemongrass | 0.2% |

### Example 13

In this example, for computing an amount of magnesium to be included in a formulation comprising the Triangle Ingredients, an estimate is used that an amount of absorbed magnesium is in a range of about 10-25%, which corresponds to an amount of magnesium applied to the skin but NOT absorbed (such as due to being washed-off) being in a range of about 75-90%.

### Example 14 (Magnesium Sulfate)

In this example, a formulation comprising magnesium sulfate is as follows:

magnesium sulfate 1-10%
water 15-20%

### FloMag® 45-55%

### Propanediol 17.5-22.5%

### LysoFix® 3.5-5%

### Example 15 (Formulation Process)

A preferred formulation process is as follows:

1) Mix/shake magnesium hydroxide continuously.

2) Mix magnesium hydroxide with water (preferably for about 5 minutes).

3) Add glycerin (and) Glycine soja (soybean) seed extract and mix (preferably for about 5 minutes).

4) Mix propanediol with acacia and xanthan gums and mix (preferably for about 5 minutes) and add to the magnesium-containing mixture and mix (preferably for about 5 minutes).

5) Add sodium bicarbonate, mix (preferably for about 5 minutes)

6) Add heptyl undecylenate (and essential oils), mix (preferably for about 10 minutes)

### Example 15A

In this example, steps 1-4 of Example 15 are performed, without performing steps 5-6.

### Example 16

A sprayable solution for transdermal magnesium delivery is prepared, comprising:

water
magnesium sulfate
propanediol
glycerin & Glycine soja (soybean)

### Example 16A

A sprayable solution for transdermal magnesium delivery according to Example 16 is prepared, containing no magnesium hydroxide.

### Example 17 (Lotion)

In this example the Triangle Ingredients are formulated as a lotion comprising 16% magnesium chloride, 16% propanediol.

### Example 18 (Lotion)

In this example, a lotion comprising magnesium chloride and propanediol is formulated without including glycerin and glycine soya extract.

### Example 19 (Lotion Formulation)

### Water 25-60%

### Magnesium Chloride 2%-30%

### Propanediol 2.5%-40%

C13-16 Isoparaffin (and) Heptyl Undecylenate 0%-20%
Glyceryl Stearate (and) Sodium Lactylate 0-15%

### Corn Starch Modified 0-5%

### Acacia Gum 0-5%

### Shea Butter—0-5%

### Cocoa Butter—0-5%

Phenylpropanol (and) propanediol (and) Caprylyl Glycol (and) Tocopherol 0-5%

### Xanthan Gum 0-5%

### Example 19A (Lotion)

### 49.8% Water

### 10% Magnesium Chloride

### 15% Propanediol

13% C13-16 Isoparaffin (and) Heptyl Undecylenate
5% Glyceryl Stearate (and) Sodium Lactylate

### 2% Corn Starch Modified

### 2% Acacia Gum

### 1% Shea Butter

### 1% Cocoa Butter

1% Phenylpropanol (and) propanediol(and) Caprylyl Glycol (and) Tocopherol

### 0.2% Xanthan Gum

### Example 19B (Lotion)

### Water 25-60%

### Magnesium Chloride 2%-30%

### Propanediol 2.5%-40%

C13-16 Isoparaffin (and) Heptyl Undecylenate 0%-20%
Glyceryl Stearate (and) Sodium Lactylate 0-15%

### Corn Starch Modified 0-5%

### Acacia Gum 0-5%

### Shea Butter—0-5%

### Cocoa Butter—0-5%

Phenylpropanol (and) propanediol(and) Caprylyl Glycol (and) Tocopherol 0-5%

### Xanthan Gum 0-5%

### Glycerin and Glycine Soja (Soybean) Seed Extract

### Example 20 (Lotion Formulation)

### Water 25-60%

### Magnesium Chloride 2%-30%

### Propanediol 2.5%-40%

C13-16 Isoparaffin (and) Heptyl Undecylenate 0-20%
Glyceryl Stearate (and) Sodium Lactylate 0-15%

### Glycerin and Glycine Soja (Soybean) Seed Extract 0.5/−7.5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Essential Oils 0-3%

### Natural Fragrance 0-3%

### Example 20A (Lotion)

### 49.8% Water

### 10% Magnesium Chloride

### 15% Propanediol

10% C13-16 Isoparaffin (and) Heptyl Undecylenate
4.5% Glyceryl Stearate (and) Sodium Lactylate

### 3.5% Glycerin and Glycine Soja (Soybean) Seed Extract

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 21 (Lotion Formulation)

### Water 25%-60%

### Magnesium Hydroxide 2%-60%

### Propanediol 2.5%-40%

C13-16 Isoparaffin (and) Heptyl Undecylenate 0-20%
Glyceryl Stearate (and) Sodium Lactylate 0-15%

### Glycerin and Glycine Soja (Soybean) Seed Extract 0.5%-7.5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Essential Oils 0-3%

### Natural Fragrance 0-3%

### Example 21A (Lotion)

### 39.8% Water

### 20% Magnesium Hydroxide

### 15% Propanediol

10% C13-16 Isoparaffin (and) Heptyl Undecylenate
4.5% Glyceryl Stearate (and) Sodium Lactylate

### 3.5% Glycerin and Glycine Soja (Soybean) Seed Extract

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 22 (Lotion Formulation)

### Water 25%-60%

### Magnesium Hydroxide 2%-60%

### Propanediol 2.5%-40%

C13-16 Isoparaffin (and) Heptyl Undecylenate 0-20%

### Magnesium Chloride 0.5-25%

Glyceryl Stearate (and) Sodium Lactylate 0-15%

### Glycerin and Glycine Soja (Soybean) Seed Extract 0.5%-7.5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Essential Oils 0-3%

### Natural Fragrance 0-3%

### Example 22A (Lotion)

### 33.8% Water

### 18% Magnesium Hydroxide

### 15% Propanediol

10% C13-16 Isoparaffin (and) Heptyl Undecylenate

### 5% Magnesium Chloride

4.5% Glyceryl Stearate (and) Sodium Lactylate

### 3.5% Glycerin and Glycine Soja (Soybean) Seed Extract

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 23 (Lotion Formulation)

### Water 25%-60%

### Magnesium Chloride 2%-30%

### Propanediol 2.5%-40%

### Capric/caprylic Triglyceride 0-10%

C13-16 Isoparaffin (and) Heptyl Undecylenate 0-10%
Glyceryl Stearate (and) Sodium Lactylate 0-10%

### Corn Starch Modified) 0-5%

### Acacia Gum 0-5%

### Shea Butter 0-5%

### Cocoa Butter 0-5%

Phenylpropanol (and) propanediol(and) Caprylyl Glycol (and) Tocopherol 0-5%

### Xanthan Gum 0-5%

### Example 23A (Lotion)

### 55.8% Water

### 15% Magnesium Chloride

### 14% Propanediol

### 5% Capric/caprylic Triglyceride

4% C13-16 Isoparaffin (and) Heptyl Undecylenate
4% Glyceryl Stearate (and) Sodium Lactylate

### 2% Corn Starch Modified)

### 2% Acacia Gum

### 1% Shea Butter

### 1% Cocoa Butter

1% Phenylpropanol (and) propanediol(and) Caprylyl Glycol (and) Tocopherol

### 0.2% Xanthan Gum

### Example 23B (Lotion)

### Water 25%-60%

### Magnesium Chloride 2%-30%

### Propanediol 2.5%-40%

### Capric/caprylic Triglyceride 0-10%

C13-16 Isoparaffin (and) Heptyl Undecylenate 0-10%
Glyceryl Stearate (and) Sodium Lactylate 0-10%

### Corn Starch Modified) 0-5%

### Acacia Gum 0-5%

### Shea Butter 0-5%

### Cocoa Butter 0-5%

Phenylpropanol (and) propanediol(and) Caprylyl Glycol (and) Tocopherol 0-5%

### Xanthan Gum 0-5%

### Glycerin and Glycine Soja (Soybean) Seed Extract

### Example 24 (Lotion Formulation)

### Water 25%-60%

### Magnesium Chloride 2%-30%

### Propanediol 2.5%-40%

### Capric/caprylic Triglyceride 0-10%

C13-16 Isoparaffin (and) Heptyl Undecylenate 0-10%

### Glycerin and Glycine Soja (Soybean) Seed Extract 0.5%-7.5%

Glyceryl Stearate (and) Sodium Lactylate 0-5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Example 24A (Lotion)

### 55.8% Water

### 15% Magnesium Chloride

### 14% Propanediol

### 5% Capric/caprylic Triglyceride

4% C13-16 Isoparaffin (and) Heptyl Undecylenate

### 2% Glycerin and Glycine Soja (Soybean) Seed Extract

2% Glyceryl Stearate (and) Sodium Lactylate

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 25 (Lotion Formulation)

### Water 25%-60%

### Magnesium Hydroxide 2%-60%

### Propanediol 2.5%-40%

### Capric/caprylic Triglyceride 0-10%

C13-16 Isoparaffin (and) Heptyl Undecylenate 0-10%

### Glycerin and Glycine Soja (Soybean) Seed Extract 0.5-7.5%

Glyceryl Stearate (and) Sodium Lactylate 0-5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Example 25A (Lotion)

### 35.8% Water

### 20% Magnesium Hydroxide

### 14% Propanediol

### 5% Capric/caprylic Triglyceride

4% C13-16 Isoparaffin (and) Heptyl Undecylenate

### 2% Glycerin and Glycine Soja (Soybean) Seed Extract

2% Glyceryl Stearate (and) Sodium Lactylate

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 26 (Lotion Formulation)

### Water 25%-60%

### Magnesium Hydroxide 2-60%

### Propanediol 2.5%-40%

Magnesium Chloride Oil (50% solids/50% water)

### Capric/caprylic Triglyceride 0-10%

C13-16 Isoparaffin (and) Heptyl Undecylenate 0-10%

### Glycerin and Glycine Soja (Soybean) Seed Extract. 5%-7.5%

Glyceryl Stearate (and) Sodium Lactylate 0-5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Example 26A (Lotion)

### 30.8% Water

### 15% Magnesium Hydroxide

### 14% Propanediol

10% Magnesium Chloride Oil (50% solids/50% water)

### 5% Capric/caprylic Triglyceride

4% C13-16 Isoparaffin (and) Heptyl Undecylenate

### 2% Glycerin and Glycine Soja (Soybean) Seed Extract

2% Glyceryl Stearate (and) Sodium Lactylate

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 27 (Cream Formulation)

### Magnesium Chloride 2%-30%

### Water 2%-30%

### Cocoa Butter 1-30%

### Propanediol 2.5%-40%

C13-16 Isoparaffin (and) Heptyl Undecylenate 0-20%

### Shea Butter 0-15%

Glyceryl Stearate (and) Sodium Lactylate 0-15%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Example 27A (Cream)

### 10% Magnesium Chloride

### 31.3% Water

### 15% Cocoa Butter

### 15% Propanediol

12% C13-16 Isoparaffin (and) Heptyl Undecylenate

### 5% Shea Butter

4.5% Glyceryl Stearate (and) Sodium Lactylate

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 28 (Cream Formulation)

### Magnesium Chloride 2-30%

### Water 2%-30%

### Cocoa Butter 1-30%

### Propanediol 2.5%-40%

C13-16 Isoparaffin (and) Heptyl Undecylenate 0-20%

### Shea Butter 0-15%

Glyceryl Stearate (and) Sodium Lactylate 0-15%

### Glycerin and Glycine Soja (Soybean) Seed Extract 0.5%-7.5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Example 28A (Cream)

### 10% Magnesium Chloride

### 29.8% Water

### 15% Cocoa Butter

### 15% Propanediol

10% C13-16 Isoparaffin (and) Heptyl Undecylenate

### 5% Shea Butter

4.5% Glyceryl Stearate (and) Sodium Lactylate

### 3.5% Glycerin and Glycine Soja (Soybean) Seed Extract

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 29 (Cream Formulation)

### Magnesium Chloride 2-30%

### Water 2-30%

### Cocoa Butter 1-30%

### Propanediol 2.5%-40%

### Shea Butter 0-10%

### Capric/caprylic Triglyceride 0-10%

C13-16 Isoparaffin (and) Heptyl Undecylenate 0-10%
Glyceryl Stearate (and) Sodium Lactylate 0-5%

### Corn Starch Modified 0-5%

### Acacia Gum 0-5%

Phenylpropanol (and) propanediol (and) Caprylyl Glycol (and) Tocopherol 0-5%

### Xanthan Gum 0-5%

### Example 29A (Cream)

### 15% Magnesium Chloride

### 36.8% Water

### 15% Cocoa Butter

### 14% Propanediol

### 5% Shea Butter

### 5% Capric/caprylic Triglyceride

4% C13-16 Isoparaffin (and) Heptyl Undecylenate
2% Glyceryl Stearate (and) Sodium Lactylate

### 2% Corn Starch Modified

### 2% Acacia Gum

1% Phenylpropanol (and) propanediol (and) Caprylyl Glycol (and) Tocopherol

### 0.2% Xanthan Gum

### Example 30 (Cream Formulation)

### Magnesium Chloride Oil 2-30%

### Water 2-30%

### Cocoa Butter 1-30%

### Propanediol 2.5%-40%

### Shea Butter 0-15%

### Capric/caprylic Triglyceride 0-10%

C13-16 Isoparaffin (and) Heptyl Undecylenate 0-10%

### Glycerin and Glycine Soja (Soybean) Seed Extract. 5%-7.5%

Glyceryl Stearate (and) Sodium Lactylate 0-5%

### Corn Starch Modified 0-5%

### Acacia Gum 0-5%

Phenylpropanol (and) propanediol (and) Caprylyl Glycol (and) Tocopherol 0-5%

### Xanthan Gum 0-5%

### Example 30A (Cream)

### 15% Magnesium Chloride Oil

### 39.8% Water

### 15% Cocoa Butter

### 14% Propanediol

### 5% Shea Butter

### 5% Capric/caprylic Triglyceride

4% C13-16 Isoparaffin (and) Heptyl Undecylenate

### 2% Glycerin and Glycine Soja (Soybean) Seed Extract

2% Glyceryl Stearate (and) Sodium Lactylate

### 2% Corn Starch Modified

### 2% Acacia Gum

1% Phenylpropanol (and) propanediol (and) Caprylyl Glycol (and) Tocopherol

### 0.2% Xanthan Gum

### Example 31 (Cream Formulation)

### Water 2-30%

### Magnesium Hydroxide 2-60%

### Cocoa Butter 0-30%

### Magnesium Chloride 0-15%

### Shea Butter 0-15%

### Propanediol 2.5%-40%

### Capric/caprylic Triglyceride 0-10%

C13-16 Isoparaffin (and) Heptyl Undecylenate 0-10%

### Glycerin and Glycine Soja (Soybean) Seed Extract 0.5%-7.5%

Glyceryl Stearate (and) Sodium Lactylate 0-5%

### Corn Starch Modified 0-5%

### Acacia Gum 0-5%

### Shea Butter 0-5%

### Cocoa Butter 0-5%

Phenylpropanol (and) propanediol (and) Caprylyl Glycol (and) Tocopherol 0-5%

### Xanthan Gum 0-5%

### Example 31A (Cream)

### 20.8% Water

### 15% Magnesium Hydroxide

### 15% Cocoa Butter

### 5% Magnesium Chloride

### 5% Shea Butter

### 14% Propanediol

### 5% Capric/caprylic Triglyceride

4% C13-16 Isoparaffin (and) Heptyl Undecylenate

### 2% Glycerin and Glycine Soja (Soybean) Seed Extract

2% Glyceryl Stearate (and) Sodium Lactylate

### 2% Corn Starch Modified

### 2% Acacia Gum

### 1% Shea Butter

### 1% Cocoa Butter

1% Phenylpropanol (and) propanediol (and) Caprylyl Glycol (and) Tocopherol

### 0.2% Xanthan Gum

### Example 32

A gel deodorant has been formulated, comprising magnesium chloride, propanediol, silica and zinc lactate.

### Example 32A

A gel deodorant has been formulated, comprising:

magnesium chloride 16%
propanediol 15%
silica 6.5%
zinc lactate 2%

### Example 33 (Formulation of Gel Deodorant)

### Water: 10-70%

### Magnesium Chloride. 5%-40%

### Propanediol: 2.5-50%

### Silica: 2-20%

### Zinc Lactate: 0-8%

### Caprylyl Glycol: 0-5%

### Phenylpropanol: 0%-5%

### Tocopherol: 0%-5%

### Ethylhexylglycerin: 0%-5%

### Essential Oils 0-4%

### Natural Fragrance 0-4%

### Example 33A (Gel Deodorant)

### 54% Water

### 16% Magnesium Chloride

### 16% Propanediol

### 6.5% Silica

### 2% Zinc Lactate

### 1% Caprylyl Glycol

### 0.5% Phenylpropanol

### 0.5% Tocopherol

### 0.5% Ethylhexylglycerin

### 3% Natural Fragrance

### Example 33B (Gel Deodorant)

### Water: 10-70%

### Magnesium Chloride 0.5%-40%

### Propanediol: 2.5-50%

### Silica: 2-20%

### Zinc Lactate: 0-8%

### Caprylyl Glycol: 0-5%

### Phenylpropanol: 0%-5%

### Tocopherol: 0%-5%

### Ethylhexylglycerin: 0%-5%

### Glycerin and Glycine Soja (Soybean) Seed Extract

### Essential Oils: 0-4%

### Natural Fragrance: 0-4%

### Example 34 (Formulation of Gel)

### Water 0-50%

### Magnesium Chloride 0.5-40%

### Propanediol 20-60%

### Sodium Hydroxide 7-10%

### Stearic Acid 5-10%

### Polyglyceryl-10 Caprylate/Caprate 0-8%

### Fragrance 0-4%

### Example 34A (Gel)

### 15% Water

### 15% Magnesium Chloride

### 45% Propanediol

### 9.5% Sodium Hydroxide

### 6% Stearic Acid

### 8% Polyglyceryl-10 Caprylate/Caprate

### 1.5% Fragrance

### Example 35 (Formulation of Gel)

### Water 0-10%

### Magnesium Chloride 0.5-40%

### Propanediol 20-70%

### Sodium Hydroxide 7-10%

### Stearic Acid 5-10%

Glycerin & glycine (soya) extract 0-5%

### Polyglyceryl-10 Caprylate/Caprate 0-8%

### Fragrance 0-4%

### Example 35A (Gel)

### 6% Water

### 10% Magnesium Chloride

### 60% Propanediol

### 9.5% Sodium Hydroxide

### 6% Stearic Acid

2% Glycerin & glycine (soya) extract

### 6% Polyglyceryl-10 Caprylate/Caprate

### 0.5% Fragrance

### Example 36 (Formulation of Gel)

### Magnesium Chloride. 55-40%

### Propanediol 20-70%

glycerin 10-19.5%
glycerin and glycine (soya) extract 0.5-10%

### Sodium Hydroxide 8-12%

### Stearic Acid 5-7.5%

### Ethylhexylglycerin 0.1-2%

### Fragrance 0-4%

### Example 36A (Gel)

### 15% Magnesium Chloride

### 45% Propanediol

15% glycerin
5% glycerin and glycine (soya) extract

### 9.5% Sodium Hydroxide

### 6% Stearic Acid

### 0.75% Ethylhexylglycerin

### 0.75% Fragrance

### Example 37 (Formulation of Gel)

### Water 0-10%

Magnesium Hydroxide slurry 2-50%

### Propanediol 30-65%

### Sodium Hydroxide 8-12%

### Stearic Acid 5-7.5%

### Polyglyceryl-10 Caprylate/Caprate 0-8%

### Sodium Bicarbonate 0-2%

### Fragrance 0-4%

### Example 37A (gel)

18% Magnesium Hydroxide slurry

### 58% Propanediol

### 9.5% Sodium Hydroxide

### 6% Stearic Acid

### 8% Polyglyceryl-10 Caprylate/Caprate

### 0.5% Fragrance

### Example 38 (Formulation of Gel)

### Water 0-20%

### Magnesium Chloride 0.5-20%

### Magnesium Hydroxide. 5-40%

### Propanediol 30-60%

glycerin 1-20%
glycerin and glycine (soya) extract 1-10%

### Polyglyceryl-10 Caprylate/Caprate 0-8%

### Sodium Hydroxide 8-12%

### Stearic Acid 5-7.5%

### Ethylhexylglycerin 0.1-2

### Fragrance 0-4%

### Example 38A (Gel)

### 7.25% Water

### 10% Magnesium Chloride

### 10% Magnesium Hydroxide

### 45% Propanediol

10% glycerin
10% glycerin and glycine (soya) extract

### 9.5% Sodium Hydroxide

### 6% Stearic Acid

### 0.75% Ethylhexylglycerin

### 0.75% Fragrance

### Example 39 (Roll-On)

A solution for use in a roll-on has been formulated, comprising:

magnesium hydroxide 42%
magnesium chloride 5%
propanediol 20%
glycerin & glycine soya oil 5%

### Example 40 (Roll-On)

When using magnesium hydroxide to formulate a solution for a roll-on product, a preferred magnesium hydroxide is a slurry that has a magnesium to water ratio of 60/40. FloMag® is the most preferred magnesium hydroxide slurry.

Ratio of total magnesium to total water in a formulation varies based on additional amount of water (if any) in the formulation from contribution other than magnesium hydroxide, as well as additional amount of magnesium (if any) in the formulation from contribution other than magnesium hydroxide (e.g., when magnesium chloride is included in a formulation along with a 60/40 magnesium hydroxide slurry).

### Example 41 (Roll-on Formulations/Gout Treatment)

### Water 2-60%

### Magnesium Hydroxide Slurry 20-80%

### Propanediol 10-50%

Glycerin (and) Glycine Soja (Soybean) Seed Extract 3.5-10%

### Heptyl Undecylenate 0-10%

### Acacia Gum 0.1-2%

### Sodium Bicarbonate 0-5%

### Xanthan Gum 0.1-1%

### Menthol 0-2%

Essential Oils (camphor, eucalyptus, rosemary, lemongrass) 0-5%
Natural Fragrance(s) (limonene, citral, citronellol, geraniol) 0-5%

### Example 41A (Roll-on/Gout Treatment)

### 13.5% Water

### 50% Magnesium Hydroxide Slurry

### 20% Propanediol

5% Glycerin (and) Glycine Soja (Soybean) Seed Extract

### 5% Heptyl Undecylenate

### 0.7% Acacia Gum

### 1% Sodium Bicarbonate

### 0.3% Xanthan Gum

### 1% Menthol

2.5% Essential Oils (camphor, eucalyptus, rosemary, lemongrass)
1% Natural Fragrance(s) (limonene, citral, citronellol, geraniol)

### Example 42 (Shave Cream)

### Water 1-70%

### Magnesium Hydroxide 2-60%

### Propanediol 1-50%

### Glycerin and Glycine Soja (Soybean) Seed Extract 0.5%-15%

### Aloe Barbadensis (Aloe Vera) Leaf Juice 0-25%

### Shea Butter 0-20%

### Coconut Oil 0-20%

### Olive Oil 0-10%

### Jojoba Oil 0-10%

### Rosemary Extract 0-5%

### Guar Gum Extract 0-5%

### Example 43 (Body Wash)

### Water 1-80%

### Magnesium Chloride 2-40%

### Glycerin and Glycine Soja (Soybean) Seed Extract 0.5%-15%

### Propanediol 1-50%

### Aloe Barbadensis (Aloe Vera) Leaf Juice 0-25%

### Shea Butter 0-20%

### Coconut Oil 0-15%

### Olive Oil 0-15%

### Jojoba Oil—0-10%

### Rosemary Extract 0-5%

### Guar Gum Extract 0-5%

### Example 44 (Body Wash)

### Water 1-80%

### Magnesium Hydroxide 2-40%

### Glycerin and Glycine Soja (Soybean) Seed Extract. 5%-15%

### Propanediol 1-50%

### Aloe Barbadensis (Aloe Vera) Leaf Juice 0-25%

### Shea Butter 0-20%

### Coconut Oil 0-15%

### Olive Oil 0-15%

### Jojoba Oil—0-10%

### Rosemary Extract 0-5%

### Guar Gum Extract 0-5%

### Example 45 (Body Wash)

### Water 1-80%

### Magnesium Sulfate 2-40%

### Glycerin and Glycine Soja (Soybean) Seed Extract 0.5%-15%

### Propanediol 1-50%

### Aloe Barbadensis (Aloe Vera) Leaf Juice 0-25%

### Shea Butter 0-20%

### Coconut Oil 0-15%

### Olive Oil 0-15%

### Jojoba Oil—0-10%

### Rosemary Extract 0-5%

### Guar Gum Extract 0-5%

### Example 46 (Cleanser)

### Water 1-80%

### Magnesium Chloride 2-40%

### Glycerin and Glycine Soja (Soybean) Seed Extract 0.5-15%

### Propanediol 1-50%

### Aloe Barbadensis (Aloe Vera) Leaf Juice 0-25%

### Shea Butter 0-20%

### Coconut Oil 0-15%

### Olive Oil 0-15%

### Jojoba Oil 0-10%

### Rosemary Extract 0-5%

### Guar Gum Extract 0-5%

### Xanthan Gum 0-5%

### Caprylic Triglyceride 0-15%

### Cetearyl Glucoside 0-15%

### Cetearyl Alcohol 0-15%

### Cetearyl Olivate 0-10%

### Denatured Alcohol 0-15%

### Sodium Hyaluronate 0-10%

### Sodium Benzoate 0-10%

### Example 47 (Cleanser)

### Water 1-80%

### Magnesium Hydroxide 2-40%

### Glycerin and Glycine Soja (Soybean) Seed Extract 0.5-15%

### Propanediol 1-50%

### Aloe Barbadensis (Aloe Vera) Leaf Juice 0-25%

### Shea Butter 0-20%

### Coconut Oil 0-15%

### Olive Oil 0-15%

### Jojoba Oil 0-10%

### Rosemary Extract 0-5%

### Guar Gum Extract 0-5%

### Xanthan Gum 0-5%

### Caprylic Triglyceride 0-15%

### Cetearyl Glucoside 0-15%

### Cetearyl Alcohol 0-15%

### Cetearyl Olivate 0-10%

### Denatured Alcohol 0-15%

### Sodium Hyaluronate 0-10%

### Sodium Benzoate 0-10%

### Example 48 (Blemish Cream)

### Water 1-80%

### Magnesium Hydroxide 1-80%

### Magnesium Chloride 0-15%

### Magnesium Sulfate 0-15%

### Propanediol 1-50%

### Glycerin and Glycine Soja (Soybean) Seed Extract. 5-7.5%

### Xanthan Gum 0-5%

### Guar Gum Extract 0-5%

### Citric Acid 0-5%

### Tea Tree Leaf Oil 0-5%

### Caprylic Triglyceride 0-5%

### Cetearyl Olivate 0-5%

### Citric Acid 0-5%

### Rosemary Leaf Oil 0-5%

### Lavender Oil 0-5%

### Sorbitan Olivate 0-5%

### Jojoba Oil 0-5%

### Menthol 0-5%

### Example 49 (Spray Deodorant)

### Water 1-80%

### Magnesium Chloride 1-30%

### Propanediol 1-50%

### Polyglyceryl-10 Caprylate/Caprate 0-10%

### Natural Fragrance 0-5%

### Example 50 (Spray Deodorant)

### Water 1-80%

### Magnesium Chloride 1-30%

### Propanediol 1-50%

### Glycerin and Glycine Soja Seed Extract 0.5-7.5%

### Polyglyceryl-10 Caprylate/Caprate 0-10%

### Natural Fragrance 0-5%

### Example 51 (Body Spray)

### Water 1-80%

Magnesium chloride (10%) 1-30%

### Propanediol 1-50%

### Ethanol (8%) 0-15%

Polysorbate 80 (bio-based version available soon) 0-10%

### Polyglyceryl-10 Caprylate/caprate 0-10%

Essential oils 0-7.5%
Soyethyl morpholinium ethosulfate 0-5%

### Example 52 (Body Spray)

### Water 1-80%

Magnesium chloride 1-30%

### Propanediol 1-50%

### Glycerin and Glycine Soja Seed Extract 0.5-7.5%

### Ethanol (8%) 0-15%

Polysorbate 80 (bio-based version available soon) 0-10%

### Polyglyceryl-10 Caprylate/caprate 0-10%

Essential oils 0-7.5%
Soyethyl morpholinium ethosulfate 0-5%

### Example 53 (Body Spray)

### Water 1-80%

Magnesium sulfate 1-30%

### Propanediol 1-50%

### Polysorbate 80 0-40%

### Ethanol 0-15%

### Polyglyceryl-10 Caprylate/caprate 0-10%

Essential oils 0-7%
Myristamidopropyl PG-dimonium chloride phosphate 0-5%

### Example 54 (Body Spray)

### Water 1-80%

Magnesium sulfate 1-30%

### Propanediol 1-50%

### Glycerin and Glycine Soja Seed Extract 0.5-7.5%

### Polysorbate 80 0-40%

### Ethanol 0-15%

### Polyglyceryl-10 Caprylate/caprate 0-10%

Essential oils 0-7%
Myristamidopropyl PG-dimonium chloride phosphate 0-5%

### Example 55 (Toothpaste)

### Water 2%-80%

Glycerin (and) Glycine Soja (Soybean) Seed Extract 0.5%-10%
Magnesium Hydroxide (60% solids/40% water) 0.5%-40%

### Propanediol (Zea Mays) 0.5%-20%

### Sorbitol—0-10%

Aloe barbadensis leaf juice (Organic)—0-10%

### Xylitol 0-5%

### Natural Flavor 0-10%

### Benzoic Acid (Natural) 0-10%

### Phosphoric Acid 0-5%

### Disodium Phosphate 0-5%

### Sodium Fluoride 0-5%

### Example 56 (Toothpaste)

### Water 2%-80%

Glycerin (and) Glycine Soja (Soybean) Seed Extract 0.5%-10%

### Magnesium Chloride 0.5%-40%

### Propanediol (Zea Mays) 0.5%-20%

### Sorbitol—0-10%

Aloe barbadensis leaf juice (Organic)—0-10%

### Xylitol 0-5%

### Natural Flavor 0-10%

### Benzoic Acid (Natural) 0-10%

### Phosphoric Acid 0-5%

### Disodium Phosphate 0-5%

### Sodium Fluoride 0-5%

### Example 57 (Foot Powder)

### 62.5% Corn Starch Modified

### 20% Avena Sativa Kernel Flour

### 5% Magnesium Oxide

### 5% Zinc Oxide

### 5% Sodium Bicarbonate

### 3% Kaolin

### 0.3% Eucalytus Oil

### 0.2% Peppermint Oil

### Example 58 (Foot Powder)

### 62.5% Corn Starch Modified

### 20% Avena Sativa Kernel Flour

### 10% Magnesium Oxide

### 5% Sodium Bicarbonate

### 3% Kaolin

### 0.3% Eucalytus Oil

### 0.2% Peppermint Oil

### Example 59

The following roll-on formulations are useable as body deodorant and/or against muscle pain.

### Example 59A

Magnesium hydroxide 45-55%

### Water 10-20%

### Propanediol 15-25%

Glycerin (and) Glycine soja (soybean) seed extract 1-10%

### Emollient 3-10%

### Menthol 5-15%

### Camphor 3-10%

### Eucalyptus 3-10%

### Witch Hazel 2-10%

### Example 59B

Magnesium hydroxide 35-45%

### Water 10-20%

### Propanediol 15-25%

Glycerin (and) Glycine soja (soybean) seed extract 1-10%

### Emollient 3-10%

### Menthol 5-15%

### Camphor 3-10%

### Eucalyptus 3-10%

### Witch Hazel 2-10%

### Aloe Vera 5-15%

### Arnica 2-6%

### Example 60 (Total Magnesium)

“Total Magnesium” refers to magnesium, by weight, contributed by all magnesium-containing ingredients in a formulation. To illustrate, in some formulations, the only magnesium-containing ingredient is a MgOH slurry. In other formulations, the only magnesium-containing ingredient is MgCl. In other formulations, the magnesium-containing ingredients include both a MgOH slurry and MgCl. In yet other formulations, another magnesium-containing ingredient, or another combination of two or more magnesium-containing ingredients, is used.

### Example 60A (Roll-Ons)

In this example, total magnesium is in a range of 0.1%-40% by weight of the roll-on solution.

### Example 60B (Roll-Ons)

In this example, total magnesium is in a range of 25-30% by weight of the roll-on solution.

### Example 60B (Roll-Ons)

In this example, total magnesium is in a range of 28-28.5% by weight of the roll-on solution.

### Example 60C (Gels)

In this example, total magnesium is in a range of 0.1%-40% by weight of the gel.

### Example 60D (Gels)

In this example, total magnesium is in a range of 10-30% by weight of the gel.

### Example 60E (Gels)

In this example, total magnesium is about 16% by weight of the gel.

### Example 60F (Lotions and Creams)

In this example, total magnesium is in a range of 0.1-60% by weight of the lotion or cream.

### Example 60G (Lotions and Creams)

In this example, total magnesium is in a range of 5-25% by weight of the lotion or cream.

### Example 60H (Lotion)

In this example, total magnesium is about 10% by weight of the lotion.

### Example 60I (Lotion)

In this example, total magnesium is about 15% by weight of the lotion.

### Example 60J (Body Wash)

In this example, total magnesium is in a range of 2-40% by weight of the body wash solution.

### Example 60K (Body Sprays and Deodorant Sprays)

In this example, total magnesium is in a range of about 1-30% by weight of the spray solution.

### Example 60L (Toothpaste)

In this example, total magnesium is in a range of about 0.5-40% by weight of the toothpaste.

### Example 60M (Foot Powder)

In this example, total magnesium is in a range of about 2-40% by weight of the foot powder.

The descriptions of various embodiments of the present invention have been presented for purposes of illustrations, but are not intended to be exhaustive. Many modifications and variations will be apparent to those of ordinary skill in the art without departing from the scope and spirit of the described embodiments.

1.-20. (canceled)

21. A lotion, comprising:
a magnesium compound selected from the group consisting of magnesium hydroxide; magnesium chloride and magnesium sulfate; and
propanediol.

22. The lotion of claim 21, further comprising glycerin and Glycine soja (soybean).

23. (canceled)
